(19) United States
(12) Patent Application Publication
(10) Pub. No.: US 2016/0272935 A1
(43) Pub. Date: Sep. 22, 2016

(54) Method of improving the yield of algae production

(71) Applicant: RELIANCE INDUSTRIES LIMITED, Mumbai, Maharashtra (IN)

(72) Inventors: Gautam Das, Andhara Pradesh (IN); Santanu Dasgupta, Maharashtra (IN); Venkatesh Prasad, Karnataka (IN); Pranali Deore, Maharashtra (IN); Deepak Nanjappa, Karnataka (IN)

(21) Appl. No.: 15/036,082

(22) PCT Filed: Nov. 12, 2014
(86) PCT No.: PCT/IN2014/000717

§ 371 Date: May 12, 2016

(30) Foreign Application Priority Data
Nov. 13, 2013 (IN) 3568/MUM/2013

(51) Int. Cl. C12N 1/12

(52) U.S. Cl. CPC C12N 1/12

(57) ABSTRACT

The present disclosure discloses a method of improving the yield of algae from an environment consisting of diatoms. This selective culturing of algae from a mixed environment is achieved by the use of a urea cycle pathway inhibitor. The inhibitor as disclosed in the present disclosure inhibits the urea cycle pathway in diatoms, thus restricting their growth so as to aid the proliferation of algae.

### FIELD

The present disclosure relates to algae, and methods of its production

### BACKGROUND

Living organisms can be divided into two groups, organism of one group employ a urea cycle pathway in their metabolism and the second group do not employ such a urea cycle pathway. The organisms which employ the urea cycle pathway in their metabolism includes diatom, ciliates, dinoflagellates, copepods and rotifers, whereas the organisms which do not employ the urea cycle pathway in their metabolism include blue-green algae, green algae and the like.

In a natural habitat algae and diatoms, both are present together in a symbiotic association. Since the algae and the diatom proliferate in the same natural ecosystem, they are difficult to separate. Both diatoms and algae utilise the same available nutrients in the ecosystem where they thrive, thus creating a competition for utilisation of nutrients. Since diatoms possess urea cycle pathways, which the algae lack, the diatoms utilize the nutrients, especially the carbon and the nitrogen sources present in the surroundings more efficiently.

As the diatom population increases, the algal population decreases. This can be countered by providing a steady supply of nutrients to the diatom-algae populated environment. But this is not a feasible option as the diatoms by virtue of the urea cycle pathway consume the nutrients more efficiently and also at a much faster rate than algae.

Therefore there is a need for a method to inhibit the growth of diatoms and at the same time increase the growth of algae. The required method needs to be moderate in cost, safe for the algal growth, efficacious and environmental friendly.

### OBJECTS

Some of the objects of the present disclosure aimed to ameliorate one or more problems of the prior art or to at least provide a useful alternative are listed herein below.

It is an object of the present disclosure to provide a method of improving the yield of algae in an environment comprising diatoms and algae.

Other objects and advantages of the present disclosure will be more apparent from the following description, which is not intended to limit the scope of the present disclosure.

### SUMMARY

Provided in the present disclosure is a method for improving the yield of algae present in a mixed environment, which includes organisms like diatoms and algae. The method as disclosed in the present disclosure inhibits the growth of diatoms whereas promotes the algal growth, in a mixed environment.

The urea cycle is a metabolic pathway used to incorporate excess nitrogen into urea and remove it from the body of an organism. However, it appears to play a far more wide-ranging role in diatoms. The urea cycle is a key to the distribution and recycling of inorganic carbon and nitrogen, and also helps diatoms recover from short-term withdrawal of nutrients.

In diatoms, the urea cycle pathway serves in the formation of carbonaceous and nitrogenous compounds and also serves as a distribution and repackaging hub for inorganic carbon and nitrogen and contributes significantly to the metabolic response of diatoms by facilitating rapid recovery from prolonged nitrogen limitation or nutrient scarcity or from short-term nutrient withdrawal and respond immediately to the availability of a greater supply of food by increasing their metabolic and growth rates. Therefore this urea cycle pathway represents a key pathway for anaplerotic carbon fixation into nitrogenous compounds that are essential for diatom growth.

### DESCRIPTION

Organisms like the diatoms which possess a urea cycle pathway are well developed to utilize rapidly nutrients, especially the carbon and nitrogen sources present, as compared to the organisms without the urea cycle like algae. Thus, in comparison the organisms like diatoms grow more rapidly, thereby shunting the growth of organisms like algae.

Therefore in accordance with the present disclosure there is provided a method of improving the yield of algae by introducing a urea cycle pathway inhibitor in a natural habitat consisting of a mixed environment of diatoms and algae.

The method involves treating the mixed environment with at least one urea cycle pathway inhibitor. The algal source can be algae grown in an open habitat.

In one embodiment the disclosed method selectively inhibits the growth of diatoms without affecting the algal growth by use of at least one urea cycle pathway inhibitor. The urea cycle pathway inhibitor used in the method of the present disclosure include but is not limited to palmitoyl CoA, tris, hepes, saccharopine, lysine, plant ornithine analogs, ammonia, sodium benzoate and combinations thereof. The disclosed method may include one or more of the aforementioned urea cycle pathway inhibitors. These urea cycle pathway inhibitors as disclosed in the present disclosure inhibit one or more of the enzymes from the group consisting of carbamoyl phosphate synthase, ornithine carbamoyl transferase, arginosuccinate synthase, arginosuccinate lyase and arginase to a greater or lesser extent. The amount of the urea cycle pathway inhibitor used is in a range of 0.1 to 100 mM with respect to the total mass of the medium. The amount of Palmitoyl coA is in the range of 0.25 mM to 0.58 mM, preferably 0.41 mM. The amount of Tris is in the range of 20 mM to 50 mM, preferably 25 mM. The amount of Saccharopine is in the range of 0.1 mM to 1 mM, preferably 0.5 mM. The amount of sodium benzoate is in the range of 1 mM to 100 mM.

In on field trials the algae population present in a habitat such as a water pond is treated with at least one urea cycle pathway inhibitor. The treatment involves techniques such as contacting, mixing, incorporating, adding, spraying and the like. The urea cycle containing organisms like diatoms are inhibited by the urea cycle pathway inhibitors, thereby enhancing the algal yield.

The disclosure will now be described with the help of the following non-limiting examples. Although the present disclosure discloses a method of improving the yield of the algae in an environment comprising of the diatoms and the algae by introducing at least one urea cycle pathway inhibitor, person ordinarily skilled in the art can explore these examples for inhibiting the growth of other organisms which employ the urea cycle.

### Example 1

A control was established containing equal quantities of algae and diatoms. Similarly a test was also established containing the same equal quantities of algae and diatoms along with 1 mM of urea cycle pathway inhibitor namely, Saccharopine.

At Day 1 using a cellometer technique the cell counts of algae and diatoms in both the control and the test was carried out. The initial cell count at day 1 for algae and diatoms were found to be 100 and 200 respectively in both the control and the test.

Both the control and the test were then incubated for 4 days in similar optimum conditions with periodic checking for cell counts in both the control and the test vessels.

The diatom and the algal cell count was then determined using the cellometer technique at the end of 4^{th} day. The algae cell count in the control was found to be 119 and that in the test was found to be 450.

On the other hand, diatom cell count of the control was found to be 842 and that of the test was found to be 62. This clearly shows that the algae cell count is not affected by the presence of the urea cycle pathway inhibitor; rather it aids the algal growth, simultaneously drastically reducing the diatom cell count from 842 to 62.

The diatom cell count in the test of the present disclosure was reduced by 69% as compared to the control as illustrated in Table 1.

TABLE 1
Cell count of Diatoms and Algae.

| Day 1 (Cell count) | | | | Day 4 (Cell Count) | | | |
|---|---|---|---|---|---|---|---|
| Control | | Inhibitor | | Control | | Inhibitor | |
| Diatom | Algae | Diatom | Algae | Diatom | Algae | Diatom | Algae |
| 200 | 100 | 200 | 100 | 842 | 119 | 62 | 450 |

### Example 2

Another experiment was carried out in a natural habitat i.e. a pond consisting of a mixed environment of algae and diatoms. At Day 1 using a cellometer technique the cell counts of algae and diatoms was carried out. The initial cell count at day 1 for algae and diatoms were found to be 1000 cells/meter and 2000 cells/meter.

A urea cycle pathway inhibitor, namely Tris 25 mM was added to the pond.

Periodic cell count readings were carried out for a span of two months. The diatom and the algal cell count were then determined using the cellometer technique at the end of the 2 month span. The algae cell count was found to be 5300 cells/meter and the diatom cell count was found to be 580 cells/meter.

This clearly shows that the algae cell count is not affected by the presence of urea cycle pathway inhibitor; rather it aids the algal growth, simultaneously drastically reducing the diatom cell count from 2000 cells/meter to 580 cells/meter.

The diatom cell count of the pond in the present disclosure was reduced by 71% as compared to the cell count of diatoms on Day, 1 as illustrated in Table 2.

TABLE 1
Cell count of Diatoms and Algae in a pond.

| Cell count at Day 1 (cells/meter) | | Cell count at Day 60 (cells/meter) | |
|---|---|---|---|
| Diatom | Algae | Diatom | Algae |
| 2000 | 1000 | 580 | 5300 |

Similar trials were also conducted using palmitoyl CoA, tris, hepes, saccharopine, lysine, plant ornithine analogs, ammonia, sodium benzoate with equally successful results.

“Whenever a range of numerical values is specified, a value up to 50% below and above the lowest and highest numerical value respectively, of the specified range, is included in the scope of the disclosure, unless there is a statement in the specification specific to the contrary”.

While considerable emphasis has been placed herein on the preferred embodiments, it will be appreciated that many embodiments can be made and that many changes can be made in the preferred embodiments without departing from the principles of the disclosure. These and other changes in the preferred embodiments as well as other embodiments of the disclosure will be apparent to those skilled in the art from the disclosure herein, whereby it is to be distinctly understood that the forgoing descriptive matter is to be implemented merely as illustrative of the disclosure and not as limitation.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Technical Advancements and Economical Significance

The technical advancements offered by the present disclosure include the realization of:
a simple and economic method which does not require highly skilled technicians for the inhibition of diatoms.
a method for inhibiting the growth of diatoms which is environmental friendly.
a method for inhibiting the growth of diatoms without adversely affecting the growth of algae.
a method for optimizing the growth of algae in a mixed environment containing diatoms.

1. A method of improving the yield of algae in an environment comprising of diatoms and algae by introducing a urea cycle pathway inhibitor to that environment.

2. A method as claimed in claim 1, wherein the urea cycle pathway inhibitor is selected from the group consisting of Palmitoyl coA, Tris, Hepes, Saccharopine, sodium benzoate, lysine, plant ornithine analogs, ammonia and combinations thereof.

3. A method as claimed in claim 1, wherein the amount of the urea cycle pathway inhibitor is introduced into that environment is in a range of 0.1 to 100 mM

4. A method as claimed in claim 2, wherein the urea cycle inhibitor is Palmitoyl coA in the range of 0.25 mM to 0.58 mM, preferably 0.41 mM.

5. A method as claimed in claim 1, wherein the urea cycle inhibitor is Tris in the range of 20 mM to 50 mM, preferably 25 mM.

6. A method as claimed in claim 1, the urea cycle inhibitor is Saccharopine in the range of 0.1 mM to 1 mM, preferably 0.5 mM.

7. A method as claimed in claim 1, the urea cycle inhibitor is sodium benzoate in the range of 1 mM to 100 mM.
